(19) United States
(12) Patent Application Publication
(10) Pub. No.: US 2017/0360718 A1
(43) Pub. Date: Dec. 21, 2017

(54) INTRANASAL DENTAL ANESTHETIC

(71) Applicant: St. Renatus, LLC, Fort Collins, CO (US)

(72) Inventor: Mark D. Kollar, Fort Collins, CO (US)

(21) Appl. No.: 15/610,915

(22) Filed: Jun. 1, 2017

Related U.S. Application Data
(60) Provisional application No. 62/343,900, filed on Jun. 1, 2016.

(51) Int. Cl. A61K 31/045; A61K 47/12; A61K 31/381; A61K 9/00; A61K 31/167; A61K 47/38; A61K 47/02

(52) U.S. Cl. CPC A61K 31/045; A61K 47/38; A61K 47/12; A61K 47/02; A61K 9/0019; A61K 31/167; A61K 9/0078; A61K 9/0043; A61K 31/381

(57) ABSTRACT

The present invention relates to intranasally administered pharmaceutical compositions administered for use in anesthesia. Such pharmaceutical compositions comprise benzyl alcohol. The invention also relates to methods for anesthetizing the maxillary dental arch using these pharmaceutical compositions.

### RELATED APPLICATION

This application claims priority to and the benefit of U.S. Provisional Application No. 62/343,900, filed Jun. 1, 2016, now expired. The entire disclosure of the above application is hereby incorporated by reference.

### TECHNICAL FIELD OF INVENTION

The present invention relates to intranasally administered pharmaceutical compositions for use in anesthesia. Such pharmaceutical compositions comprise benzyl alcohol. The invention also relates to methods for anesthetizing the maxillary dental arch using these pharmaceutical compositions.

### BACKGROUND OF THE INVENTION

The most common means for anesthetizing maxillary teeth is the use of an infiltration injection. For example, the patient may receive a needle penetration and injection of a local anesthetic solution at the approximate apex of the root on the buccal surface of a tooth (or teeth) to be treated. A maxillary palatal injection of dental local anesthetic can be used for regional block anesthesia of teeth. The patient may also receive a maxillary palatal injection of local anesthesia in order to effect regional nerve blocks.

Dental anxiety, including needle or shot discomfort and phobia, is a long-standing barrier to regular dental care. In a survey conducted by the American Dental Association, 27% of the people surveyed indicated “fear of pain” as a reason to avoid dental visits. Needle injection of local anesthetic is recognized as the most anxiety-provoking procedure in dentistry for children and adults. Not only can the injection of local anesthetics produce fear, pain and an increase in blood pressure, but the increased stress of injection can result in fainting, hyperventilation, convulsions, shock, hypertension, cardiac arrest, respiratory collapse, acute allergic reaction to preservatives, bronchospasm, angina, and organ system toxicity in medically compromised patients. Further, local injections into target nerves can sometimes lead to permanent numbness as a result of needles damaging and/or cutting nerves. In rare cases, a severe overdose of injectable anesthetic or accidental, rapid intravascular injection of the anesthetic can even lead to death. The act of injecting is invasive (conceptually, psychologically, and physically) and is a major reason worldwide why people avoid regular dental care and see their dentists only in an emergency. In fact, some studies indicate that millions of people in the US are afraid of dental treatments because of pain. Without regular dental care, tooth caries, gum disease and oral cancer may be left undetected and can result in infection and serious, systemic health issues.

In addition, the risk of exposure to bloodborne pathogens via needlesticks is a recognized occupational hazard in dentistry. Since 1991, the Occupational Safety and Health Administration (“OSHA”) has enforced a Bloodborne Pathogens Standard that requires employers to identify, evaluate and implement safer medical devices to eliminate or minimize employee exposure to bloodborne pathogens. The standard was revised in 2001 when Congress passed the Needlestick Safety and Prevention Act. The Act set forth in greater detail employers' obligations under the Bloodborne Pathogens standard and specifically indicated that a “safer medical device” includes needleless systems. Thus, further incentive exists to develop anesthetics that can be delivered without the use of needles and comply with U.S. federally mandated engineered work practice controls.

Lidocaine, the most widely used local dental anesthetic today, was first introduced in the 1940s. Despite many attempts at developing needle-free anesthetic, the only widely-used non-injectable, subsurface local anesthetic commercially available today is Oraqix® gel. While Oraqix® can be used in periodontal pockets during scaling or root-planing procedures, it does not provide adequate anesthesia for use in procedures such as filling, crowns and root canals. Further, while many advances in the comfort of dentistry have been made, such as behavior modification therapy, topical numbing around the injection site, nitrous oxide, hypnosis, and TENS units, none of these advances has eliminated the need for needle injection.

Nasal administration of anesthetics has also been disclosed, for example, in U.S. Pat. No. 6,413,499.

Thus, there remains a need for alternative and effective anesthetics. In particular, there remains a need for alternative and effective anesthetics and means for administering such anesthetics to patients who do not respond to local anesthetics (e.g., articaine).

### SUMMARY OF THE INVENTION

The present invention provides intranasally administered pharmaceutical compositions comprising benzyl alcohol and methods of use thereof.

In one embodiment, the intranasal pharmaceutical composition comprises benzyl alcohol. In a particular embodiment, the intranasal pharmaceutical composition of the present invention comprises the components, and respective amounts, listed in Table 1 below (referred to herein as the “Table 1 Pharmaceutical Composition.”)

TABLE 1

| | Pharmaceutical |
|---|---|
| Ingredient | Composition (% w/v) |
| Benzyl alcohol, NF | 0.90 |
| Citric acid anhydrous, USP | 1.00 |
| Sodium hydroxide, NF | q.s. |
| Hydrochloric acid, NF | q.s. |
| Hydroxyethylcellulose, NF (5000 cps) | 0.05 |
| Purified water, USP | q.s. to 100% |

Particular non-limiting embodiments of the invention are set forth in the following numbered paragraphs

1. A method for anesthetizing at least a portion of a patient's maxillary dental arch of a subject comprising administering to said subject an intranasal pharmaceutical composition comprising:
a) benzyl alcohol; and
b) a pharmaceutically acceptable carrier, wherein benzyl alcohol is the sole active agent in said intranasal pharmaceutical composition.

2. The method of paragraph 1, further comprising administering to said subject a local anesthetic.

3. The method of paragraph 1 or 2, wherein the subject has not responded to local anesthetic previously.

4. The method of any one of paragraphs 1-3, wherein said intranasal pharmaceutical composition comprises about 0.6-1.2% (w/v) benzyl alcohol.

5. The method of paragraph 4, wherein said intranasal pharmaceutical composition comprises about 0.75-1.05% (w/v) benzyl alcohol.

6. The method of paragraphs 5, wherein said intranasal pharmaceutical composition comprises about 0.9% (w/v) benzyl alcohol.

7. The method of any one of paragraphs 1-6, wherein said intranasal pharmaceutical composition further comprises a preservative.

8. The method of paragraph 7, wherein said preservative is selected from the list consisting of: sugar alcohols, ethanol, benzyl alcohol, isopropanol, cresol, chlorocresol, and phenol.

9. The method of any one of paragraphs 1-8, wherein said intranasal pharmaceutical composition further comprises a viscosity enhancing agent and/or penetration enhancer agent.

10. The method of paragraph 9, wherein said viscosity enhancing agent is selected from the list consisting of: methylcellulose, hydroxyethylcellulose, hydroxypropylmethylcellulose and smart hydrogel.

11. The method of paragraph 11, wherein said viscosity enhancing agent is hydroxyethylcellulose.

12. The method of paragraph 11, wherein said intranasal pharmaceutical composition comprises about 0.01-1.0% (w/v) hydroxyethylcellulose.

13. The method of paragraph 12, wherein said intranasal pharmaceutical composition comprises about 0.05% (w/v) hydroxyethylcellulose.

14. The method of any one of paragraphs 1-13, wherein said pharmaceutically acceptable carrier is water.

15. The method of any one of paragraphs 1-14, wherein said intranasal pharmaceutical composition has a pH of 4.0-6.5.

16. The method of paragraph 15, wherein said intranasal pharmaceutical composition has a pH of 5.5-6.5.

17. The method of any one of paragraphs 1-16, wherein said intranasal pharmaceutical composition comprises:

| | Pharmaceutical |
|---|---|
| Ingredient | Composition (% w/v) |
| Benzyl alcohol, NF | 0.90 |
| Citric acid anhydrous, USP | 1.00 |
| Sodium hydroxide, NF | q.s. |
| Hydrochloric acid, NF | q.s. |
| Hydroxyethylcellulose, NF (5000 cps) | 0.05 |
| Purified water, USP | q.s. to 100% |

18. The method of any one of paragraphs 1-17, wherein said local anesthetic is selected from lidocaine or articaine.

19. The method of any one of paragraphs 1-18, wherein at least a portion of the intranasal pharmaceutical composition is absorbed by nasal tissue located at the rear of the nasal cavity in the vicinity of the anterior dental plexus, extensions of the middle superior nerve, the posterior superior alveolar nerve, the nociceptors of the facial and buccal nerve, the sphenopalatine (pterygopalatine) ganglion or the trigeminal ganglion.

20. The method of any one of paragraphs 1-19, wherein said intranasal pharmaceutical composition is delivered by nebulization or spraying.

21. The method of paragraph 20, wherein said delivered spray is a stream or a plume.

22. The method of paragraph 21, wherein the selective delivery into the nasal cavity, nasal sinuses or rear of the nasal cavity comprises spraying said intranasal pharmaceutical composition 1-5 times into each of the nostrils of said subject.

23. The method of paragraph 22, wherein said selective delivery into the nasal cavity, nasal sinuses or rear of the nasal cavity comprises spraying said intranasal pharmaceutical composition 3 times into each of the nostrils of said subject.

24. The method of paragraph 22 or 23, wherein each of the sprays into each of the nostrils of said subject are separated by an interval of about 4 minutes.

25. The method of any one of paragraphs 1-24, wherein about 1.8 mg of benzyl alcohol is delivered into the nasal sinuses or rear of the nasal cavity of said subject.

26. The method of any one of paragraphs 1-25, wherein about 5.4 mg of benzyl alcohol is delivered into the nasal sinuses or rear of the nasal cavity of said subject.

27. The method of any one of paragraphs 1-26, wherein the particle size of said intranasal pharmaceutical composition that is delivered to the nasal cavity, nasal sinuses or rear of the nasal cavity is about 5-50 microns.

28. The method of any one of paragraphs 1-27, wherein the particle size of said intranasal pharmaceutical composition is about 10-20 microns.

29. The method of any one of paragraphs 1-28, wherein the particle size of said intranasal pharmaceutical composition is about 10 microns or larger.

30. The method of any one of paragraphs 1-29, wherein at least 85% of the particles of the intranasal pharmaceutical composition are about 10 microns or larger.

31. The method of any one of paragraphs 2-30, wherein said local anesthetic is administered via injection.

32. The method of paragraph 31, wherein said local anesthetic is administered via local infiltration or as a nerve block 33. The method of any one of paragraphs 2-32, wherein said local anesthetic is administered after the intranasal pharmaceutical composition.

34. The method of paragraph 33, wherein said intranasal treatment is a pre-treatment before the use of the local anesthetic.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The singular forms “a”, “an”, and “the” include the plurals unless the context clearly indicates otherwise.

In order to further define the invention, the following terms and definitions are provided herein.

### Definitions

“Anesthesia success” or “anesthetic success” as used herein refers to the use of anesthesia on a subject without the need for rescue anesthesia at the time of the dental procedure. “Anesthesia failure” or “anesthetic failure” as used herein refers to the use of anesthesia on a subject that requires rescue anesthesia due to a lack of anesthesia at the time of the dental procedure.

“Anesthesia rescue” or “rescue” as used herein refers to a further attempt at anesthesia in a subject that has undergone anesthesia failure using a local anesthetic.

“Anesthetizing” as used herein refers to administering an anesthetic compound (e.g., benzyl alcohol) for the purpose of preventing sensation and/or reducing pain.

“Benzyl alcohol” as used herein refers to the organic compound with the formula C₆H₅CH₂OH. Benzyl alcohol is also referred to as “phenylmethanol,” “phenol carbinol,” “phenylcarbinol,” “benzenemethanol,” “alpha-hydroxytoluene,” “phenyl carbinol,” and “phenylmethyl alcohol.”

“Intranasal formulation” as used herein refers to a formulation that is delivered to the nasal cavity and/or nasal sinuses.

“Local anesthetic” as used herein refers to an anesthetic delivered near the nerve ending to be anesthetized which diffuses after introduction inside the tissue. A local anesthetic is capable of penetrating the tissue deep enough so as to reach the target nerves and nerve branches. A local anesthetic is different from a “topical anesthetic” in that a topical anesthetic only provides surface anesthesia to a depth of approximately 1-2 mm. A topical anesthetic does not penetrate deep enough in the tissue to reach nerve branches or teeth.

“Maxilla” as used herein refers to either of a pair of bones of the human skull fused to form the upper jaw. The maxilla is sometimes simply referred to as the upper jaw.

“Maxillary dental arch” or “maxillary arch” as used herein refers to the curved structure formed by the maxillary (upper) teeth in their natural position.

“Mucosal tissue” or “mucous tissue” as used herein refers to a type of epithelial tissue. Mucosal tissue refers to the layer of tissue which covers the surface of organs or lines a cavity. Non-limiting examples of mucosal tissue include, tissue in the ear, throat, mouth, eye, nose, rectal area and/or the urogenital tract (such as the vagina).

“Non-responder” or “subject that has not responded to local anesthetic previously” as used herein refers to a subject that has on at least one previous occasion had anesthesia failure with a local anesthetic. The non-response may be as reported by the subject or documented in the subject's dental records.

“Pulpal anesthesia” as used herein refers to a level of anesthesia such that when a electric pulp testing (EPT) stimulation (i.e., a reading of 65 on the pulp tester) is administered to a tooth, the subject indicates no pain sensation in the tooth.

“Preceding nerves” as used herein refers to nerve endings and nerves located upstream, usually present in a more central neural bundle, from a particular reference nerve.

“Subject” as used herein refers to an animal including a mammal (e.g., a human).

“Tetracaine” (sometimes referred to as “amethocaine”) as used herein refers to (2-(dimethylamino)ethyl 4-(butylamino)benzoate) and any salt thereof. Tetracaine HCl refers to the HCl salt of 2-(dimethylamino)ethyl 4-(butylamino)benzoate.

### Pharmaceutical Compositions

The present invention provides intranasal benzyl alcohol based anesthetic formulations. These formulations are useful for anesthetizing at least a portion of a body part, organ, or tissue. In one aspect, the present invention provides an intranasal pharmaceutical composition for anesthetizing at least a portion of a body part, organ, or tissue by administering the intranasal pharmaceutical composition of the invention.

In some embodiments, the target tissue is epithelial tissue. In other embodiments, the target tissue is mucosal tissue. In yet other embodiments, the mucosal tissue is a tissue or membrane present in the mouth (e.g., gums).

In some embodiments, the present invention uses local anesthetics in combination with the intranasal benzyl alcohol pharmaceutical compositions of the disclosure. Local anesthetics are known to those of skill in the art. Non-limiting examples of local anesthetics include ester-based anesthetics such as, tetracaine cocaine, procaine, chloroprocaine and benzocaine; and amide-based local anesthetics, such as articaine, lidocaine, mepivacaine, bupivacaine, etidocaine, prilocaine, and ropivacaine. In some embodiments, local anesthetics are formulated for delivery by injection. In some embodiments, anesthesia by local anesthetics is via nerve block or via infiltration.

Any vasoconstrictor may be used in the pharmaceutical composition of this invention. The vasoconstrictors used in the pharmaceutical compositions of the invention has several purposes. First, the vasoconstrictor shrinks the tissue (e.g., nasal tissue) thereby enhancing penetration of the anesthetic into the tissue (e.g., deeper into the nasal cavity). Second, once the vasoconstrictor has been absorbed into the desired area, it inhibits systemic migration of the anesthetic, thereby allowing the anesthetic to remain at a desired site. Third, the vasoconstrictor reduces blood flow to the desired tissue, thereby increasing the duration of the anesthetic's effectiveness (typically by activating G-protein coupled receptors which render metallic ionophores in the nerve membrane more sensitive). Typical vasoconstrictors useful in the pharmaceutical compositions of this invention include but are not limited to indirect α₁-adrenergic agonists such as phenylephrine; calcium channel blockers; meperidine; imidazole drugs such as oxymetazoline and xylometazoline; α₂-adrenergic agonists such as guanfacine; imidazoline (I₁) ligands; direct α₂-adrenergic agonists such as clonidine; substance P blockers/reducers such as capsaicin; I-menthol; icilin; and glutamate receptor inhibitors; or pharmaceutically acceptable salts thereof. In some embodiments, the vasoconstrictor includes but is not limited to oxymetazoline, xylometazoline, guafacine, clonidine, phenylephrine, meperidine, capsaicin; I-menthol; icilin and pharmaceutically acceptable salts thereof. In some embodiments, the vasoconstrictor is oxymetazoline, or a pharmaceutically acceptable salt thereof. In other embodiments, the oxymetazoline is an oxymetazoline HCl salt.

The concentration of vasoconstrictor used in the pharmaceutical composition of the instant invention will depend on the specific vasoconstrictor and the desired vasoconstricting effect. Typical ranges of concentrations include but are not limited to about 0.01-5% (w/v). In some embodiments, the composition comprises about 0.01-1.0% (w/v) (e.g., oxymetazoline HCl salt). In yet other embodiments, the composition comprises about 0.05% (w/v) (e.g., oxymetazoline HCl salt).

In addition to its vasoconstricting activity, oxymetazoline has been shown to have a degree of anesthetic affect. Oxymetazoline affects G-protein coupled receptors and, in turn, K⁺, Na⁺, and Ca²⁺ channels present in nerve cells. In particular, oxymetazoline enhances K⁺, Na⁺, and Ca²⁺ permeability such that the nerve is no longer polarized and, thus, is unable to fire and transmit pain impulses.

In some embodiments, the intranasal pharmaceutical composition of this invention further comprises a preservative. Examples of preservatives include, but are not limited to sugar alcohols (e.g., sorbitol and mannitol), ethanol, benzyl alcohol, isopropanol, cresol, chlorocresol, phenol and benzalkonium chloride (BAK).

The concentration of preservative used in the intranasal pharmaceutical composition of the instant invention will depend on the specific preservative. Typical ranges of concentrations of preservatives are about 0.1-5% (w/v). In some embodiments, the composition comprises about 0.5-2.0% (w/v). In other embodiments, the composition comprises about 0.9% (w/v) (e.g., benzyl alcohol).

The anesthetic, vasoconstrictor and other agents used in the intranasal compositions of the present invention may be dispersed in an appropriate carrier in order to control the dosage, absorption rate and other desired properties. Pharmaceutically acceptable carriers useful in the present invention include but are not limited to aqueous carriers, gel carriers, emulsifiers, surfactants, time release vehicles, nanoparticles, microspheres, intra- and para-cellular transport chemicals, polymers, and chitin. Examples of aqueous carriers include, but are not limited to, water, buffers (e.g., phosphate buffer, citrate buffer), sugar alcohols, alcohols such as ethanol, or any other solvent that is biologically compatible with the target tissue.

The pH of the intranasal pharmaceutical composition of the present invention is typically adjusted to be compatible with the pH of the tissue to which it is being administered (e.g., the nasal cavity). Typically, the composition has a pH of about 4.0-6.5. In some embodiments, the composition has a pH of about 5.5-6.5. In further embodiments, the intranasal pharmaceutical composition has a pH of about 6.0 to about 6.5. The skilled artisan will appreciate that higher or lower pHs than those recited may be required and would readily know how to adjust the pH accordingly.

In some embodiments, the intranasal pharmaceutical composition of this invention further comprises a viscosity enhancing agent. Examples of viscosity enhancing agents include, but are not limited to methylcellulose, hydroxyethylcellulose, hydroxypropylmethylcellulose and smart hydrogel. In some embodiments, the viscosity enhancing agent is hydroxyethylcellulose. In some embodiments, the intranasal pharmaceutical composition of this invention further comprises a penetration enhancer agent.

Viscosity enhancing agents aid in controlling the mobility of the formulation before, during, and after application of the formulation to a target body part, tissue or organ. For example, viscosity enhancing agents aid in controlling the amount of formulation that either drips out of the nostrils or into the throat of the subject by reducing the mobility of the applied formulation. Viscosity enhancing agents also increase the amount of time the formulation is in contact with the vasculature (e.g., nasal vasculature) which helps to increase the uptake efficiency of the formulation to the vasculature. In addition, viscosity enhancing agents further dilute moieties present in the nasal passage that degrade esters (e.g., esterase) and can be mildly cilio-inhibitory.

The concentration of viscosity enhancing agent used in the intranasal pharmaceutical composition of the instant invention will depend on the specific viscosity enhancing agent. Typical ranges of viscosity enhancing agents, include 0.01 to 5% (w/v). In some embodiments, the intranasal pharmaceutical composition comprises about 0.01-1.0% (w/v). In other embodiments, the composition comprises about 0.05% (w/v) (e.g., hydroxyethylcellulose). The inventors have observed that compositions comprising high amounts of viscosity enhancing agents (e.g., hydroxyethylcellulose) result in clogging of the spray apparatus (e.g., the BD Accuspray™ device).

In a particular embodiment, the intranasal pharmaceutical composition of the present invention is the Table 1 formulation. In another embodiment, the Table 1 formulation is for intranasal administration.

In some embodiments, the pH of the intranasal pharmaceutical composition is between about 4.0 to about 7.5. In other embodiments, the pH of the intranasal pharmaceutical composition is between about 4.0 to about 6.5. In another embodiment the intranasal pharmaceutical composition has a pH of about 5.5 to about 6.5. In further embodiments, the intranasal pharmaceutical composition has a pH of about 6.0 to about 6.5.

In other embodiments, the intranasal pharmaceutical compositions optionally comprise pharmaceutically acceptable excipients. Examples of pharmaceutically acceptable excipients include, but are not limited to, preservatives, surfactants, stabilizing agents, emulsifiers, antibacterial agents, buffering agents and viscosity modifying agents. Specific examples of such excipients are discussed above.

Examples of emulsifiers include, but are not limited to, acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxypropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine and xanthan gum.

### Methods of Anesthetizing Tissue

In some embodiments, the invention provides a method for anesthetizing at least a portion of a patient's maxillary dental arch (and pain fibers external to the dental arch) in a subject comprising selectively delivering into the nasal cavity of the subject an intranasal pharmaceutical composition of the invention, wherein at least a portion of the intranasal pharmaceutical composition is absorbed by nasal tissue located at the rear of the nasal cavity in the vicinity of the sphenopalatine ganglion and, consequently, the maxillary nerve (and/or structures that communicate with the maxillary nerve), thereby anesthetizing at least a portion of the maxillary dental arch (and pain fibers outside but around the maxillary arch of the subject) of the subject. In some embodiments, the intranasal pharmaceutical compositions is delivered into the nasal sinuses and/or rear of the nasal cavity of the subject. In some embodiments, the maxillary dental arch is anesthetized without producing facial numbness of the eye, surface of the nose, cheeks or lips. In some embodiments, the tissues around the maxilla (e.g., mucosal tissue such as gums) are also anesthetized. In some embodiments, the intranasal pharmaceutical composition is delivered by nebulization or spraying. In some embodiments, the intranasal pharmaceutical composition is delivered to the nasal sinuses such as the maxillary sinus. In other embodiments, the intranasal pharmaceutical composition is delivered to the anterior dental plexus, extensions of the middle superior nerve, the posterior superior alveolar nerve, the nociceptors of the facial and buccal nerve, and/or the sphenopalatine (pterygopalatine) ganglion. In some embodiments, the intranasal pharmaceutical composition is also delivered to parts of the trigeminal ganglion. In other embodiments, the delivered spray is a stream or a plume.

In order to anesthetize the maxillary dental arch, the intranasal pharmaceutical composition must be administered to the tissue located at the extreme rear upper end of the nasal cavity such that the composition is absorbed in the region of the maxillary nerve and the pterygopalatine ganglion. The maxillary nerve as used herein refers to region of the nasal cavity where the posterior, middle and anterior superior alveolar nerves collect. These nerves are responsible for the initial transmission of transmitting pain, temperature and pressure impulses generated by the nerves of the maxillary dentition and surrounding maxillary bone. After the impulses pass through the maxillary nerve and the main trunk of the trigeminal nerve, they are transmitted to the brain for processing and recognition.

Any delivery device may be used in the methods of the present invention. Examples of delivery devices suitable for intranasal administration include, but are not limited to squeeze bottles, syringes, pneumatically pressurized devices, pump sprayers, positive pressure nebulizers and the like. In some embodiments, the device used is pre-filled with a pharmaceutical composition of the present invention. In another embodiment, the device used is a BD Accuspray™ device.

In some embodiments, the method comprises spraying the intranasal pharmaceutical composition 1-5 times into each of the nostrils of the subject. In other embodiments, the method comprises spraying the intranasal pharmaceutical composition 3 times into each of the nostrils of the subject.

Without being bound by any particular theory, it is believed that the each spraying may play a unique role in the onset of the anesthetic effect. For example, in embodiments wherein the intranasal pharmaceutical composition is delivered in 3 sprays, it is believed that the first spray begins to neutralize nasal enzymatic pathways (e.g., esterases, cytochrome P450, aldehyde dehydrogenase), which may breakdown the delivered pharmaceutical composition. It is also believed that the first spray begins to paralyze the nasal cilia which may otherwise clear the composition to, for example, the pharynx. It is further believed that the composition delivered in the second and third sprays bring on the anesthetic effect.

In some embodiments, each of the sprays are administered within about 1-10 minutes of each other. For example, spray #1 may be administered, followed by an interval of about 1-10 minutes, after which spray #2 is administered, followed by an interval of about 1-10 minutes, after which spray #3 is administered, etc. In some embodiments, the interval between sprays is about 1-6 minutes. In some embodiments, the interval between sprays is about 2-6 minutes. In other embodiments, the interval between sprays is about 3-5 minutes. In yet other embodiments, the interval between sprays is about 4 minutes.

The intranasal pharmaceutical compositions of the present invention are delivered to the maxillary sinus and the anterior dental plexus, as well as extensions of the middle superior alveolar nerve and sphenopalatine ganglion. Further, the intranasal pharmaceutical compositions of the present invention anesthetize at least a portion of the branches of the maxillary nerve and the pterygopalatine ganglion. In some embodiments, the intranasal pharmaceutical composition is delivered to parts of the trigeminal ganglion (e.g., V2-V3 of the trigeminal ganglion). Depending on the extent of anesthetization, it is possible to anesthetize part or all of the surrounding tissues of the maxillary arch. In certain embodiments, the intranasal pharmaceutical compositions of the present invention anesthetize the lower teeth.

In embodiments where the method comprises spraying the intranasal pharmaceutical composition into the subject's nose, it has been found that the angle of spraying aids in targeting the formulation to the relevant portions of the nasal cavity described above. For example, where the intranasal pharmaceutical composition is delivered in 3 sprays, the following procedure may be used: For the first spray, the tip of the spray device can be positioned at an angle of about 35 to 45 degrees from the horizontal plane while the subject looks straight ahead. For the second spray, the tip of the spray device can be positioned at an angle of about −10 to +5 degrees from the horizontal plane while the subject looks straight ahead. The third spray can be delivered in the same fashion as the first spray. In some embodiments, the tip of the spray device can be positioned at an angle of about 42 degrees from the horizontal plane for the first and third sprays, and can be positioned at an angle of about zero degrees for the second spray. In other embodiments, the tip of the spray device is positioned approximately 0-2 mm inside the nose during sprayings.

In some embodiments, the targeting of specific teeth is desired. Thus, further refinements in the positioning of the spray device, in addition to those described above, may be employed. Without being bound by the following techniques, it is believed that the following modes of administration may also be employed. To target the anterior teeth (i.e., teeth 6-11), the spray device is angled about 0 to 15 degrees toward the midsagittal plane. To target the bicuspids (i.e., teeth 4, 5, 12, and 13), the spray device is angled about 5 degrees away to about 5 degrees toward the midsagittal plane. To target the posterior teeth (i.e., the molars or teeth 1-3 and 14-16), the spray device is angled about 0 to 10 degrees away from midsagittal plane. In other embodiments, the spray device is angled about 5 to 10 degrees toward the midsagittal plane for the anterior teeth, about parallel to the midsagittal plane for the bicuspids, and about 5 degrees away from midsagittal plane for the posterior teeth.

Tooth targeting may be further refined by varying the depth at which the spray device is placed within the nostril. For example, when spraying, the spray device can be placed about 2-3 mm within the nostril to target the anterior teeth, about 5 mm within the nostril to target the bicuspids, and about 7-9 mm to target the posterior teeth.

In some embodiments, a known (e.g., metered) amount of benzyl alcohol is delivered into the nasal cavity of the subject. In other embodiments, the known (e.g., metered) amount of benzyl alcohol is delivered into the nasal sinuses and/or rear of the nasal cavity of the subject. Typically, the amount of benzyl alcohol delivered to the nasal sinuses and/or rear of the nasal cavity of the subject is about 5.4 mg. In some embodiments, about 4.5, 3.6, 2.7, 1.8, or 0.9 mg of benzyl alcohol is delivered into the nasal sinuses and/or rear of the nasal cavity of the subject. In some embodiments, the intranasal composition comprises about 0.6-1.2% (w/v) benzyl alcohol. In some embodiments, the intranasal composition comprises about 0.75-1.05% (w/v) benzyl alcohol. In other embodiments, the intranasal composition comprises about 0.8-1.0% (w/v) benzyl alcohol. In yet other embodiments, an intranasal composition comprising about 0.85-0.95% (w/v) benzyl alcohol is used. In a particular embodiment, the intranasal composition comprises about 0.9% (w/v) benzyl alcohol.

In some embodiments, the particle size of the intranasal pharmaceutical composition delivered to the nasal sinuses and/or rear of the nasal cavity is about 5-50 microns. In other embodiments, the particle size of the intranasal pharmaceutical composition is about 10-20 microns. In some embodiments, the intranasal pharmaceutical composition is delivered in a manner such that at least 50% of the particles are at least about 10 microns or larger. In other embodiments, the intranasal pharmaceutical composition is delivered in a manner such that at least 65% of the particles are at least about 10 microns or larger. In other embodiments, the intranasal pharmaceutical composition is delivered in a manner such that at least 85% of the particles are at least about 10 microns or larger. In yet other embodiments, the intranasal pharmaceutical composition is delivered in a manner such that at least 90% of the particles are at least about 10 microns or larger. In further embodiments, the intranasal pharmaceutical composition is delivered in a manner such that at least 95% of the particles are at least about 10 microns or larger.

In certain embodiments, the intranasal compositions are used in combination with local anesthetics. In certain embodiments, the subject is known to be a non-responder to local anesthetics. In certain embodiments, the intranasal compositions are used as pre-treatments for subjects known to be non-responder to local anesthetics. In certain embodiments, subjects experience anesthesia failure of a local anesthetic and are rescued with the intranasal composition disclosed herein. In certain embodiments, subjects experience anesthesia failure of the intranasal composition disclosed herein and are rescued with a local anesthetic.

In certain embodiments, the local anesthetic and the intranasal composition are delivered at approximately the same time. In some embodiments, the intranasal composition is delivered before the local anesthetic. In other embodiments, the local anesthetic is delivered before the intranasal composition. In certain embodiments, the time between the delivery of the local anesthetic and the intranasal composition is about 1, 5, 10, 15, 20, 25, 30, 35, 40, 45, 50, 55, or 60 minutes or more.

In embodiments involving the combination of the intranasal compositions with local anesthetics, delivery of local anesthetics is well understood by one of ordinary skill in the art. Any delivery device may be used to apply the local anesthetic. Examples of suitable delivery devices include, but are not limited to syringes, swabs and the like. In some embodiments, the device used is pre-filled with a local anesthetic.

These and other embodiments of the invention may be further illustrated in the following non-limiting Examples.

### EXAMPLES

### Example 1: Evaluation of Local Anesthetic in Combination with Intranasal Table 1 Formulation or Tetracaine/Oxymetazoline for Anesthetizing Maxillary Teeth in Healthy Subjects

### Overview of Procedure

A study using a randomized, double-blind, parallel-groups design comparing two study drugs A and B is conducted in adult patients. Study drug A contains the intranasal tetracaine/oxymetazoline formulation described in Table 2. Study drug B contains the intranasal benzyl alcohol formulation described in Table 1. At the treatment session, subjects were randomized to one of two groups for study drug administration as follows: Group 1: tetracaine/oxymetazoline; Group 2: benzyl alcohol. 2 or 3 intranasal sprays of 200 μL each of study drug are administered on the same side as the treatment tooth. The first 2 sprays are administered at baseline (T0) and then 4 minutes later (T4). At T14, the adequacy of dental pulpal anesthesia is assessed and the Study Dental Procedure commenced at T15 if the patient reports no pain. If the patient reports pain, a third spray of study drug is administered, and 10 minutes are allowed to lapse for development of the full effect of the third dose of study drug to begin the procedure again at T25. If dental pulpal anesthesia is still not sufficient at or after T25, rescue injection of local anesthetic is administered and the dental procedure completed.

TABLE 2

| Ingredient | Formulation (% w/v) |
|---|---|
| Tetracaine hydrochloride, USP | 3.00 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Hydroxyethylcellulose, NF (5000 cps) | 0.05 |
| Purified water, USP | q.s. to 100% |

A similar study is conducted in pediatric patients.

The rescue anesthetic in the adult studies is 4% articaine hydrochloride with 1:100,000 epinephrine injection (Septocaine, Septodont). The rescue therapy in the pediatric studies is of 2% lidocaine.

### Administration of Treatment

For each spray, the subject is sitting up with his/her chin slightly down. During each spray, the subject is instructed to hold his/her breath. After each spray the subject is asked to gently sniff.

For the first spray, the tip of the spray device is positioned approximately 0-2 cm inside the nose at an angle of 0 degrees from the horizontal plane; the subject looks straight ahead. For the second and third sprays, the tip of the spray device is positioned approximately 0-2 cm inside the nose at an angle of 42 degrees from the horizontal plane as illustrated in the diagram below; again the subject looks straight ahead.

### Results

The results of one adult study is as follows:

TABLE 3
Subgroup Analysis of Success Rates by Number of Sprays (mITT)

| | Anesthetic Success Rate | | | | |
|---|---|---|---|---|---|
| | Tetracaine/ | | | | |
| | Oxymetazoline | | BA | | |
| Stratification | (N = 100) | | (N = 50) | | P- |
| Stratum | N | Count (%) | N | Count (%) | value^{a} |
| Number of sprays | | | | | |
| 2 | 73 | 72 (98.6%) | 17 | 14 (82.4%) | NA |
| 3 | 27 | 16 (59.3%) | 33 | 0 (0.0%) | |

The results in Table 3 are also summarized as follows:

TABLE 4
Success Rates by Treatment Group (mITT)

| | Tetracaine/Oxymetazoline | | BA | | |
|---|---|---|---|---|---|
| | (N = 100) | | (N = 50) | | |
| Anesthetic | Count | | Count | | |
| outcome | (%) | 95% CI | (%) | 95% CI | |
| Success | 88 | (80.0, 93.6%) | 14 | (16.2, 42.5%) | <0.0001 |
| | (88.0%) | | (28.0%) | | |
| Failure | 12 | | 36 | | |
| | (12.0%) | | (72.0%) | | |

* One-sided Fisher's Exact Test at 2.5% type-1 error

The results of another adult study is as follows:

TABLE 5
Anesthetic Success Rate by Treatment Groups (mITT Population)

| | Patient Count (Percent) By Treatment | | | | | |
|---|---|---|---|---|---|---|
| | Tetracaine/ | | | | | |
| | Oxymetazoline | | TTC | | BA | |
| | (N = 44) | | (N = 44) | | (N = 22) | |
| Anesthetic | Count | 95% | Count | 95% | Count | 95% |
| Response | (%) | CI | (%) | CI | (%) | CI |
| Success | 37/44 | (69.9%, | 12/44 | (15.0%, | 6/22 | (10.7%, |
| | (84.1%) | 93.4%) | (27.3%) | 42.8%) | (27.3%) | 50.2%) |
| Failure | 7/44 | — | 32/44 | — | 16/22 | — |
| | (15.9%) | | (72.7%) | | (72.7%) | |

| | |
|---|---|
| P-value | <0.0001 |
| Tetracaine/ | |
| Oxymetazoline | |
| vs. TCC* | |
| P-value | <0.0001 |
| Tetracaine/ | |
| Oxymetazoline | |
| vs. BA* | |

TTC: Tetracaine alone
*One-sided Fisher's Exact Test at 2.5% Type-1 Error

The adult patients treated with tetracaine/oxymetazoline indicated as failures in the above studies (18) are then treated with a local anesthetic (4% articaine). Of those, 17 are rescued (Tables 3-5). The adult dose tetracaine/oxymetazoline/LA rescue success is 94.1%.

In the benzyl alcohol/LA (BA) group there are 52 adult BA failures rescued (Tables 3-5; BA). The adult dose BA/LA rescue success is 100%.

The results of one pediatric study is as follows:

TABLE 6
Summary of Success Rates by Site and Site Dosage Cohort and Age
Group (mITT)

| | | | | | One-Sided | |
|---|---|---|---|---|---|---|
| | | | | | Fischer's | Stratified |
| | Tetracaine/Oxymetazoline | | BA | | Exact Test | CMH |
| Site/Cohort | (N = 60) | | (N = 30) | | P-Value | P-Value |
| Site A (N = 48) | 24/32 | (75.0%) | 6/16 | (37.5%) | 0.014 | 0.024 |
| Site B (N = 42) | 22/28 | (78.6%) | 10/14 | (71.4%) | 0.44 | |
| By Site and Dosage | | | | | | |
| Cohort | | | | | | |
| Site A | | | | | | |
| 10 to <20 kg | 10/12 | (83.3%) | 5/6 | (83.3%) | 0.73 | 0.0087 |
| 20 to <40 kg | 10/16 | (62.5%) | 1/8 | (12.5%) | 0.027 | |
| 40 kg or more | 4/4 | (100.0%) | 0/2 | (0.0%) | 0.067 | |
| Site B | | | | | | |
| 10 to <20 kg | 4/4 | (100.0%) | 2/2 | (100.0%) | ND | 0.61 |
| 20 to <40 kg | 4/8 | (50.0%) | 4/4 | (100.0%) | 1.00 | |
| 40 kg or more | 14/16 | (87.5%) | 4/8 | (50.0%) | 0.069 | |
| By Site and Age | | | | | | |
| Group | | | | | | |
| Site A | | | | | | |
| 3 to 5 years | 13/17 | (76.5%) | 5/7 | (71.4%) | 0.59 | 0.025 |
| 6 to 11 years | 7/11 | (63.6%) | 1/8 | (12.5%) | 0.037 | |
| 12 to 17 years | 4/4 | (100.0%) | 0/1 | (0.0%) | 0.20 | |
| Site B | | | | | | |
| 3 to 5 years | 4/4 | (100.0%) | 3/3 | (100.0%) | ND | 0.59 |
| 6 to 11 years | 7/12 | (58.3%) | 5/7 | (71.4%) | 0.86 | |
| 12 to 17 years | 11/12 | (91.7%) | 2/4 | (50.0%) | 0.14 | |

ND = not determined.

The results of another pediatric study is as follows:

TABLE 7
Incidence (N, %) of Tetracaine/Oxymetazoline Success Rate by
Cohort (mITT Population)

| Anesthetic | Cohort A | | | Cohort B | | Cohort C |
|---|---|---|---|---|---|---|
| Assessment | 1.2 mL | 2.0 mL | 4.0 mL | 1.2 mL | 2.0 mL | 1.2 mL |
| Result | (n = 8) | (n = 8) | (n = 8) | (n = 8) | (n = 8) | (n = 8) |
| Success | 6 | 7 | 8 (100.0%) | 5 | 7 | 4 |
| | (75.0%) | (88.0%) | | (63.0%) | (88.0%) | (50.0%) |
| Failure | 2 | 1 | 0 (0.0%) | 3 | 1 | 4 |
| | (25.0%) | (13.0%) | | (38.0%) | (13.0%) | (50.0%) |

In the pediatric (pedo) dosing groups there are 14 tetracaine/oxymetazoline rescued and 0 failures (Tables 6-7). The pediatric dose tetracaine/oxymetazoline/LA rescue success is 100%.

In the benzyl alcohol/LA (BA) group there 14 pediatric failures (Table 6; BA). The pediatric dose BA/LA rescue success is 100%.

In both the tetracaine/oxymetazoline/LA studies and BA/LA studies, the rescue success was unexpectedly high. Historically, the average local anesthesia success is only 71% (see 2014-CE-Course-Handout-Budenz-SA12-Are-You-Numb-Yet-Part-2-Techniques_1). Pre-treatment with either intranasal formulation appears to significantly increase the success rate over the historical success rate.

1. A method for anesthetizing at least a portion of a patient's maxillary dental arch of a subject comprising administering to said subject an intranasal pharmaceutical composition comprising:
a) benzyl alcohol; and
b) a pharmaceutically acceptable carrier,
wherein benzyl alcohol is the sole active agent in said intranasal pharmaceutical composition.

2. The method of claim 1, further comprising administering to said subject a local anesthetic.

3. The method of claim 1, wherein the subject has not responded to local anesthetic previously.

4. The method of claim 1, wherein said intranasal pharmaceutical composition comprises about 0.6-1.2% (w/v) benzyl alcohol.

5. The method of claim 4, wherein said intranasal pharmaceutical composition comprises about 0.75-1.05% (w/v) benzyl alcohol.

6. The method of claim 5, wherein said intranasal pharmaceutical composition comprises about 0.9% (w/v) benzyl alcohol.

7. The method of claim 1, wherein said intranasal pharmaceutical composition further comprises a preservative.

8. The method of claim 7, wherein said preservative is selected from the list consisting of: sugar alcohols, ethanol, benzyl alcohol, isopropanol, cresol, chlorocresol, and phenol.

9. The method of claim 1, wherein said intranasal pharmaceutical composition further comprises a viscosity enhancing agent and/or penetration enhancer agent.

10. The method of claim 9, wherein said viscosity enhancing agent is selected from the list consisting of: methylcellulose, hydroxyethylcellulose, hydroxypropylmethylcellulose and smart hydrogel.

11. The method of claim 11, wherein said viscosity enhancing agent is hydroxyethylcellulose.

12. The method of claim 11, wherein said intranasal pharmaceutical composition comprises about 0.01-1.0% (w/v) hydroxyethylcellulose.

13. The method of claim 12, wherein said intranasal pharmaceutical composition comprises about 0.05% (w/v) hydroxyethylcellulose.

14. The method of claim 1, wherein said pharmaceutically acceptable carrier is water.

15. The method of claim 1, wherein said intranasal pharmaceutical composition has a pH of 4.0-6.5.

16. The method of claim 15, wherein said intranasal pharmaceutical composition has a pH of 5.5-6.5.

17. The method of claim 1, wherein said intranasal pharmaceutical composition comprises:

| | Pharmaceutical |
|---|---|
| Ingredient | Composition (% w/v) |
| Benzyl alcohol, NF | 0.90 |
| Citric acid anhydrous, USP | 1.00 |
| Sodium hydroxide, NF | q.s. |
| Hydrochloric acid, NF | q.s. |
| Hydroxyethylcellulose, NF | 0.05 |
| (5000 cps) | |
| Purified water, USP | q.s. to 100% |

18. The method of claim 2, wherein said local anesthetic is lidocaine or articaine.

19. The method of claim 1, wherein at least a portion of the intranasal pharmaceutical composition is absorbed by nasal tissue located at the rear of the nasal cavity in the vicinity of the anterior dental plexus, extensions of the middle superior nerve, the posterior superior alveolar nerve, the nociceptors of the facial and buccal nerve, the sphenopalatine (pterygopalatine) ganglion or the trigeminal ganglion.

20. The method of claim 1, wherein said intranasal pharmaceutical composition is delivered by nebulization or spraying.

21. The method of claim 20, wherein said delivered spray is a stream or a plume.

22. The method of claim 21, wherein the selective delivery into the nasal cavity, nasal sinuses or rear of the nasal cavity comprises spraying said intranasal pharmaceutical composition 1-5 times into each of the nostrils of said subject.

23. The method of claim 22, wherein said selective delivery into the nasal cavity, nasal sinuses or rear of the nasal cavity comprises spraying said intranasal pharmaceutical composition 3 times into each of the nostrils of said subject.

24. The method of claim 22, wherein each of the sprays into each of the nostrils of said subject are separated by an interval of about 4 minutes.

25. The method of claim 1, wherein about 1.8 mg of benzyl alcohol is delivered into the nasal sinuses or rear of the nasal cavity of said subject.

26. The method of claim 1, wherein about 5.4 mg of benzyl alcohol is delivered into the nasal sinuses or rear of the nasal cavity of said subject.

27. The method of claim 1, wherein the particle size of said intranasal pharmaceutical composition that is delivered to the nasal cavity, nasal sinuses or rear of the nasal cavity is about 5-50 microns.

28. The method of claim 1, wherein the particle size of said intranasal pharmaceutical composition is about 10-20 microns.

29. The method of claim 1, wherein the particle size of said intranasal pharmaceutical composition is about 10 microns or larger.

30. The method of claim 1, wherein at least 85% of the particles of the intranasal pharmaceutical composition are about 10 microns or larger.

31. The method of claim 1, wherein said local anesthetic is administered via injection.

32. The method of claim 31, wherein said local anesthetic is administered via local infiltration or as a nerve block

33. The method of claim 1, wherein said local anesthetic is administered after the intranasal pharmaceutical composition.

34. The method of claim 33, wherein said intranasal treatment is a pre-treatment before the use of the local anesthetic.
